(19) United States
(12) Patent Application Publication
(10) Pub. No.: US 2007/0208267 A1
(43) Pub. Date: Sep. 6, 2007

(54) DEVICE FOR ACOUSTICALLY REPRODUCING THE RESPIRATORY FUNCTION FOR A RESPIRATOR

(75) Inventors: Robert SCHMID, Luebeck (DE); Ralf HEESCH, Luebeck (DE)

Correspondence Address: MCGLEW & TUTTLE, PC, P.O. BOX 9227, SCARBOROUGH STATION, SCARBOROUGH, NY, 10510-9227, US

(73) Assignee: DRAEGER MEDICAL AG & CO. KG, Luebeck (DE)

(21) Appl. No.: 11/608,961

(22) Filed: Dec. 11, 2006

(30) Foreign Application Priority Data
Mar. 4, 2006 (DE) 10 2006 010 008.5

(51) Int. Cl. A61B 5/08

(52) U.S. Cl. 600/529

(57) ABSTRACT

A device is provided for acoustically reproducing the respiratory function for a respirator, with a patient respiration circuit. The device has a noise generator, a volume flow sensor device, which determines the volume flow in the patient respiration circuit and generates an electric signal representing the volume flow, and a volume flow evaluating unit, which is set up to pick up the volume flow signal and to generate a control signal for the noise generator as a function of this in such a way that an acoustic signal with a loudness rising (falling) with rising (falling) volume flow is generated. To optimize the loudness of the acoustic signal, a microphone and an evaluating unit connected thereto are provided, which said evaluating unit is set up to generate at least a first measure for the instantaneous ambient noise level on the basis of the microphone signal by averaging over at least one period of time and to increase (reduce) the loudness of the acoustic signal with rising (falling) ambient noise level as a preset monotonic function of the at least one measure.

### CROSS REFERENCE TO RELATED APPLICATIONS

This application claims the benefit of priority under 35 U.S.C. § 119 of German Patent Application DE 10 2006 010 008.5 filed Mar. 4, 2006, the entire contents of which are incorporated herein by reference.

### FIELD OF THE INVENTION

The present invention pertains to a device for acoustically reproducing the respiratory function for a respirator (also known as ventilator) with a patient respiration circuit, the device having a noise generator, a volume flow sensor device, which determines the volume flow in the patient respiration circuit and generates an electric signal representing the volume flow, a volume flow evaluating unit, which is set up to pick up the volume flow signal and to generate, as a function thereof, a control signal for the noise generator in such a way that an acoustic signal with a loudness rising/falling with rising/falling volume flow is generated.

### BACKGROUND OF THE INVENTION

There usually are many visual displays, which display the status of the patient or his therapy, in the field of medical engineering, especially in intensive care medicine with the use of respirators including anesthesia apparatus and patient monitors. However, the therapist is not continuously able to see this information because many activities must also be carried out outside the area in which the displays are visible. Acoustic information may be useful in this case, because these can be perceived in a broader area.

The acoustic displays used most frequently are alarms, which signal a state of error or an exceptional state. If a medical parameter or a device parameter leaves a range set in advance, predefined acoustic messages are sent. A likewise widespread application is the acoustic representation of body functions, for example, the acoustic reproduction of electrocardiogram (ECG) activities (heartbeat).

Furthermore, there are devices that acoustically represent the respiration of a patient in connection with respirators. One example of such a device, which has the features as discussed in the introduction above, is integrated in the “APOLLO™” respirator of Dräger Medical AG & Co. KG. A noise generator in this device generates a noise, which, reproduced via a loudspeaker, is very similar to a stethoscope noise. The loudness of the noise generated is proportional to the volume flow in the respiration circuit, which is measured by means of a volume flow sensor device. Inspiration and expiration are also distinguished here by a low-pass filter, which is connected into the circuit during expiration, as a result of which the noise sounds higher during inspiration and deeper during expiration.

A device for the acoustic reproduction of physiological or medical data appears from U.S. Pat. No. 5,730,140.

One problem of such acoustic monitoring devices is that the basic loudness must be set by the user. This setting may subsequently prove to be too faint during phases with more ambient noise (hectic activity in the operating room, noises of other devices, etc.) and too loud during quieter phases (for example, during the operation).

### SUMMARY OF THE INVENTION

The object of the present invention is to improve a device for acoustically reproducing the respiratory function for a respirator such that the acoustic reproduction is more easily perceptible for the medical staff under changing ambient conditions.

According to the invention, a device is provided for acoustically reproducing the respiratory function for a respirator, with a patient respiration circuit. The device includes a noise generator and a volume flow sensor device, which determines the volume flow in the patient respiration circuit and generates an electric signal representing the volume flow. A volume flow evaluating unit is set up to pick up the volume flow signal and to generate, as a function of the volume flow signal, a control signal for the noise generator in such a way that an acoustic signal with a loudness rising or falling with a rising or falling volume flow is generated. A microphone, whose output signal is sent to an evaluating unit and is processed there by averaging over at least one first period of time in order to generate at least one first measure for the instantaneous ambient noise level, is provided according to the present invention in the device. The evaluating unit is set up, furthermore, to vary the loudness of the acoustic signal for displaying the volume flow as a preset monotonic (for example, proportional) function of the at least one measure for the ambient noise level such that the loudness of the acoustic signal rises (falls) with rising (falling) ambient noise level.

By determining the ambient noise level, the monitoring device can adapt the loudness of the acoustic signal to the ambient situation. The ambient noise detected with the microphone is averaged now over a first period of time in order to obtain a measure for the ambient noise level as a result. It is ensured as a result that individual, brief noise will not cause any appreciable change in the loudness.

In an advantageous embodiment, the evaluating unit is set up such that the microphone signal is averaged over a first period of time and over a second period of time different therefrom in order to obtain a first measure and a second measure for the ambient noise level. For example, the first period of time may be one second and the second period of time may be 10 seconds. The evaluating unit is then set up to select the smaller of the first measure and the second measure and to set the loudness of the noise generator in accordance with this. It is achieved as a result that a brief rise in the ambient noise level, which markedly increases the first measure but has a much lower effect in the second measure due to the further averaging, will not have any noticeable effect on the loudness of the acoustic signal generated due to the selection of the second measure. It is achieved as a result, for example, that a very brief noise, as generated by a falling medical instrument or the like, will not cause any appreciable change in the loudness. On the other hand, it is achieved that the control unit will follow the ambient noise level relatively rapidly in case of falling ambient noise level, because the measure that is obtained from the averaging over the shorter period of time is decisive now, so that the device can rapidly follow a falling ambient noise level by reducing the loudness of the acoustic signal generated.

The ambient noise level shall be determined preferably without being affected by the noise level generated by the device itself. This happens, for example, by the microphone signals being evaluated during periods only during which the evaluating unit generates no signal via the noise generator, i.e., during the breathing spaces, or they are evaluated only during phases during which no acoustic alarm is signaled. As an alternative, since the control signal for the noise generator is known, the acoustic signal generated by the noise generator can be taken into account and compensated by calculation in the measured noise signal in the evaluating unit.

A frequency range in which typical ambient noises occur, for example, a frequency range of 500 to 4,000 Hz, is preferably selected for the evaluation, and the ambient noise level is determined by the microphone in this frequency range only in order to leave irrelevant frequencies, for example, those due to structure-borne noise, out of consideration.

Respiration is also increasingly applied in the area of anesthesia in the form of assisted forms of respiration. It is often necessary here to assess the patient's own respiratory activity. However, the patient's own respiratory activity is difficult to recognize in the volume flow-pressure curve, especially in the case of pressure-supported forms of respiration, because modern respirators respond increasingly rapidly to the patient's demand. It is therefore often difficult for the therapist to determine whether a breathing stroke was applied mandatorily (because the patient did not demand a breathing stroke for too long) or whether a supported, spontaneously demanded breathing stroke was applied. Modern respirators therefore have optical displays, which signal the type of respiratory activity (spontaneous, spontaneous supported or mandatory). In an advantageous embodiment of the present invention, the type of the respiratory activity is likewise signaled acoustically. This can be carried out by varying the frequency of the acoustic respiratory flow signal generated by the noise generator in accordance with the detected type of respiratory activity. As an alternative, a special signal tone may be generated depending on the type of the respiratory activity.

The present invention will be described below on the basis of an exemplary embodiment. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which the preferred embodiment of the invention is illustrated.

### BRIEF DESCRIPTION OF THE DRAWINGS

In the drawings:

FIG. 1, is a block diagram of the device according to the invention.

### DESCRIPTION OF THE PREFERRED EMBODIMENTS

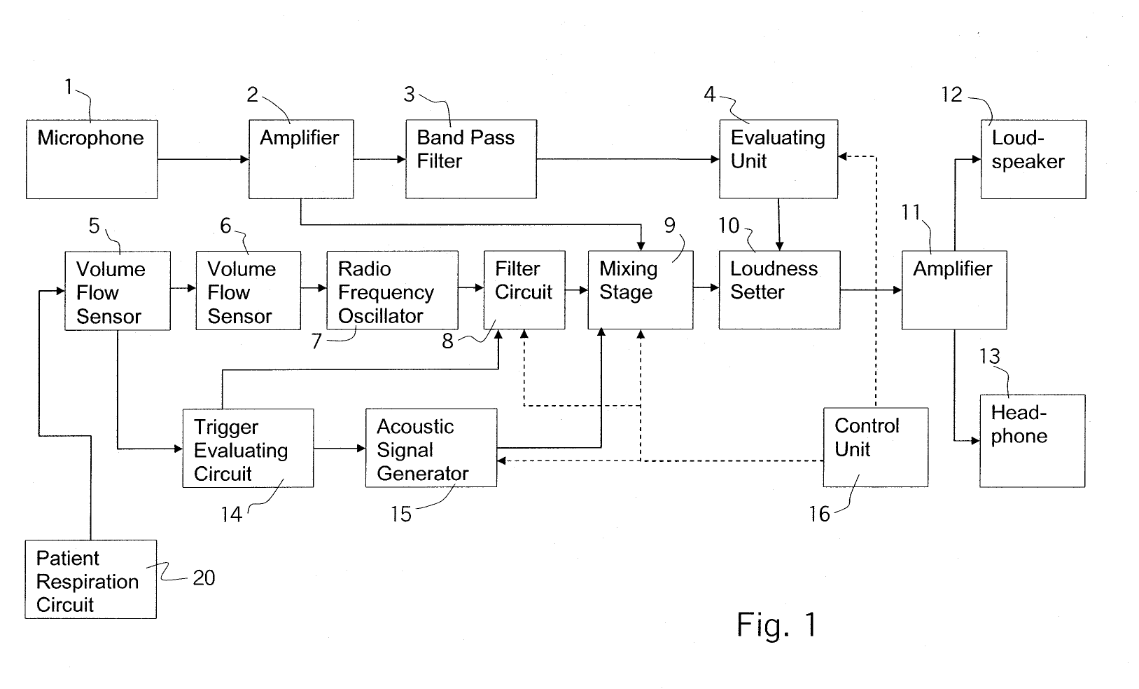

Referring to the drawings in particular, the device shown in FIG. 1 has a volume flow sensor device 5, 6, for example, by means of pressure measurement at the patient respiration circuit 20 with subsequent evaluation, which determines the patient's volume flow in the respiration circuit and generates a corresponding control signal, which is sent to a noise generator 7, which generates, via a loudspeaker 12 and/or headphone 13, a stethoscope-like respiratory sound with a loudness that depends on the respiratory volume flow.

Furthermore, a microphone 1 is provided, which detects the ambient noise level. The ambient noise level is preferably evaluated in a limited frequency range only, which comes into consideration for typical areas of the environment. This frequency range may be, for example, between 500 Hz and 4,000 Hz. The microphone signal is sent to an evaluating unit 4 via an amplifier 2 and optionally a band pass filter 3. The microphone signal is averaged in the evaluating unit 4 at least over a first period of time in order to thus obtain a first measure (or value) for the ambient noise level. As a preset, monotonic function of this measure, for example, as a proportional function, a loudness setter 10 is actuated by the evaluating unit 4. The loudspeaker 12 and/or the headphone 13 is then operated via an amplifier 11.

Averaging of the microphone signal is preferably performed in the evaluating unit 4 over a first period of time and over a second, longer period of time in order to thus obtain a first measure and a second measure for the ambient noise level. The first or second measure with the smaller value is then selected in order to set the loudness setter 10 in accordance therewith. It is achieved by this evaluation that brief increases in the ambient noise level, as they occur, for example, in case of a falling object, will not have an appreciable effect on the setting of the loudness setter 10, because these have an increasing effect only in the measure that is averaged over the shorter period of time, but they have hardly any effect on the measure obtained on the basis of the averaging over the longer period of time. It is achieved, on the other hand, that the device will adapt itself rapidly to a falling ambient noise level.

The ambient noise level is preferably determined such that the acoustic respiratory volume flow signal generated by the device itself has no effect on it. This can be achieved, for example, by the ambient noise level being evaluated during periods of time only during which there is no respiratory activity or it is evaluated only during the phases during which no acoustic alarm is signaled. As an alternative, since the actuation of the loudness setter 10 is known in the evaluating unit 4, the internal noise is compensated in the evaluating unit 4 by calculation.

The value thus determined for the ambient noise level can be used in the overall device to control additional acoustic signals (for example, the alarm loudness, the pulse tone loudness, the ECG tone loudness), and the loudness must not drop below a minimum loudness preset by the central control unit 16 in case of safety-relevant parameters (e.g., alarm).

Furthermore, a trigger evaluating circuit 14 is provided, which detects the type of the respiratory activity (spontaneous, spontaneous supported, mandatory) and sends this information to the filter circuit 8 and to an acoustic signal generator 15. The acoustic signal generator 15 thus generates, at the beginning of a breathing stroke, corresponding to the type of respiratory activity detected, an acoustic signal preset by a central control unit 16. As an alternative or in addition, the type of the detected respiratory activity may vary the respiratory volume flow signal via the filter circuit 8 depending on the detected type of respiratory activity.

The signal sent by the filter circuit 8 passes through a mixing stage 9, in which the acoustic signals from the acoustic signal generator 15, displaying the respiratory activity, are optionally superimposed. Via the loudness setter 10, which is actuated by the evaluating unit 4, the control signal then reaches the amplifier 11, whose output controls the loudspeaker 12 and is optionally or complementarily also reproduced via a headphone 13.

In order not to uncouple the therapist from the ambient noise in case of operation with a headphone, the ambient noise can be mixed with the acoustic signal after the output of the amplifier 2 in the mixing stage 9 according to the preset/set values of the central control unit 16.

Furthermore, a higher-level central control unit 16 may be provided, which can carry out additional control functions. For example, the control signal for the loudness setter 11, which is generated in the evaluating unit 4 as a function of the microphone signal, can be generated according to different, presettable functions in order to thus generate different signal intensities and/or signal patterns, for example, for premature or newborn babies, for adults, for example, depending on the sex, age and/or the body weight of the patient, and this variation can be controlled by the control unit 16. The control unit 16 may also affect the filter circuit 8 for these purposes in order to vary the acoustic signal to be generated in a preset manner.

While a specific embodiment of the invention has been shown and described in detail to illustrate the application of the principles of the invention, it will be understood that the invention may be embodied otherwise without departing from such principles.

What is claimed is:

1. A device for acoustically reproducing the respiratory function for a respirator, with a patient respiration circuit, the device comprising:
a noise generator;
a volume flow sensor device, which determines the volume flow in the patient respiration circuit and generates an electric signal representing the volume flow;
a volume flow evaluating unit, which is set up to pick up the volume flow signal and to generate, as a function of the volume flow signal, a control signal for the noise generator in such a way that an acoustic signal with a loudness rising or falling with a rising or falling volume flow is generated;
a microphone; and
an evaluating unit connected to said microphone, said evaluating unit generating a measure for an instantaneous ambient noise level on the basis of a microphone signal by averaging over at least one period of time and to increase or reduce the loudness of the acoustic signal with rising or falling ambient noise level as a preset monotonic function of said measure.

2. A device in accordance with claim 1, wherein said evaluating unit generates, on the basis of the microphone signal, said measure by averaging over a first period of time and generates another measure for the instantaneous ambient noise level by averaging over a second period of time that is different from said first period of time and selects the smaller of the first and second measures and varies the control signal as a preset monotonic function of the selected measure, so that the loudness of the acoustic signal rises or falls with a rising or falling ambient noise level.

3. A device in accordance with claim 1, wherein said evaluating unit varies the control signal in proportion to the measure.

4. A device in accordance with claim 1, wherein said noise generator has a noise generator, which is designed to simulate breath sounds like those audible with a stethoscope.

5. A device in accordance with claim 1, wherein a low-pass filter is provided as a filter circuit following said noise generator, said volume flow sensor device and a trigger evaluating circuit cooperating to provide a direction of volume flow for distinguishing the phase of inspiration from the phase of expiration and controlling the low-pass filter in accordance with the direction of flow such that the noise passing through during inspiration sounds differently and/or is distinguishable from the noise passing through during expiration.

6. A device in accordance with claim 1, wherein the noise passing through during inspiration sounds higher in pitch from the noise passing through during expiration.

7. A device in accordance with claim 1, wherein said evaluating unit evaluates a microphone signal, for generating a measure for the ambient noise level, only during periods of time during which no acoustic signal is generated by said noise generator or during periods of time during which no acoustic alarm is signaled.

8. A device in accordance with claim 1, wherein said evaluating unit compensates for a contribution to the microphone signal, which contribution is generated by said noise generator based on a correction, which is generated as a preset function of the control signal for said noise generator, in order to obtain the measure for the ambient noise level as a result.

9. A device in accordance with claim 1, wherein an input device is present to enable the basic loudness of the acoustic signal generated to be selected manually.

10. A device in accordance with claim 8, wherein said input device is designed such that the loudness cannot drop below a preset minimal basic loudness for safety-relevant acoustic indicators.

11. A device in accordance with claim 1, wherein said evaluating unit suppresses increased ambient noise levels, which do not exceed a preset maximum duration.

12. A device in accordance with claim 1, further comprising a sensor unit detecting a type of the respiratory activity as spontaneous, spontaneous supported or mandatory and that is set up, furthermore, to acoustically reproduce the type of the respiratory activity.

13. A device in accordance with claim 12, wherein said sensor unit detecting a type of the respiratory activity cooperates with a trigger evaluating circuit to vary the acoustic breathing signal in accordance with the detected type of respiratory activity.

14. A device in accordance with claim 12, wherein said trigger evaluating circuit varies the frequencies of the acoustic breathing signal generated in accordance with the detected type of respiratory activity by actuating a filter circuit.

15. A device in accordance with claim 12, wherein said trigger evaluating circuit generates a separate characteristic tone signal via a signal generator for each detected type of respiratory activity.

16. A device in accordance with claim 1, further comprising a band-pass filter to detect the ambient noise level only in a preset frequency range.

17. A device in accordance with claim 1, wherein the respirator is an intensive care respirator, an emergency respirator or anesthesia apparatus.

18. A combination respirator with device for acoustically reproducing the respiratory function, the combination comprising:
a patient respiration circuit;
a volume flow sensor determining a volume flow in said patient respiration circuit and generating an electric signal representing the volume flow;
a noise generator;
a volume flow evaluating unit for receiving said volume flow signal and generating, as a function of said volume flow signal, a control signal for the noise generator;
a microphone; and
an evaluating unit connected to said microphone, said evaluating unit generating an instantaneous ambient noise level value on the basis of a microphone signal by averaging over at east one period of time and increasing or decreasing a loudness of the output of said noise generator.

19. A device in accordance with claim 18, wherein said evaluating unit generates, on the basis of the microphone signal, said noise level value by averaging over a first period of time and generates another noise level value for the instantaneous ambient noise level by averaging over a second period of time that is different from said first period of time and selects the smaller of the first and second noise level value and varies the control signal as a function of the selected noise level value such that the loudness of an acoustic signal based on the output of the noise generator rises or falls with a rising or falling ambient noise level.

20. A device in accordance with claim 18, further comprising a low-pass filter following said noise generator, said volume flow sensor device and a trigger evaluating circuit cooperating to provide a direction of volume flow for distinguishing the phase of inspiration from the phase of expiration and controlling the low-pass filter in accordance with the direction of flow such that the noise passing through during inspiration sounds differently and/or is distinguishable from the noise passing through during expiration.

21. A device for acoustically reproducing the respiratory function for a respirator, with a patient respiration circuit, the device comprising:
a noise generator;
a volume flow sensor device, which determines the volume flow in the patient respiration circuit and generates an electric signal representing the volume flow;
a volume flow evaluating unit, which is set up to pick up the volume flow signal and to generate, as a function of the volume flow signal, a control signal for the noise generator in such a way that an acoustic signal with a loudness rising or falling with a rising or falling volume flow is generated;
a microphone;
an evaluating unit connected to said microphone, said evaluating unit generating a measure for an instantaneous ambient noise level on the basis of a microphone signal by averaging over at least one period of time and to increase or reduce the loudness of the acoustic signal with rising or falling ambient noise level as a preset monotonic function of said measure; and
a mixing stage for mixing ambient noise from said microphone with said acoustic signal to form a mixed signal.

22. A device in accordance with claim 21, further comprising an amplifier receiving an ambient noise signal from said microphone to provide an amplified ambient noise signal, said mixing stage receiving said acoustic signal and said amplified ambient noise signal to form said mixed signal.

23. A device in accordance with claim 22, further comprising headphones for providing a audible output based on said mixed signal.
